(19) United States
(12) Patent Application Publication
(10) Pub. No.: US 2009/0182049 A1
(43) Pub. Date: Jul. 16, 2009

(54) Pharmaceutical Composition and Method for Treating Hypertriglyceridemia and Hypercholesterolemia in Humans

(76) Inventor: Joar Arild Opheim, Aptos, CA (US)

Correspondence Address: JONES DAY (for Nordic Naturals), 222 EAST 41ST. STREET, NEW YORK, NY, 10017-6702, US

(21) Appl. No.: 12/015,488

(22) Filed: Jan. 16, 2008

(51) Int. Cl. A61K 31/202; A61P 3/00

(52) U.S. Cl. 514/560

(57) ABSTRACT

A method for the treatment or prophylaxis of hypertriglyceridemia and hypercholesterolemia without concomitantly increasing LDL-serum cholesterol, in a human subject requiring such treatment, which method comprises orally administering to the patient an effective amount of a pharmaceutical composition in which the active ingredients comprise a mixture of fatty acids, wherein said mixture comprises at least about 60% by weight a combination of eicosapentaenoic acid (EPA) and docosahexaenoic acid (DHA) in a weight ratio of EPA:DHA of from about 1.4:1 to about 5:1, wherein said combination is at least about 60% in the triglyceride form of the fatty acids and the balance is at least about 80% of mono and di-glycerides.

### BACKGROUND OF THE INVENTION

1. Field of the Invention

The invention relates to compositions and methods for prophylaxis and treatment of hypertriglyceridemia and hypercholesterolemia in humans.

2. Description of the Prior Art

Omega-3 fatty acids are primarily derived from fish oils and are well known to reduce serum triglycerides¹ and adverse coronary events in humans. The principal active ingredients in fish oil are eicosapentaenoic acid (EPA) and docosahexaenoic acid (DHA), which were given in one trial in a combined dose of 4 g/day for seven months to hypertriglyceridemic patients resulting in a reduction of 47% in triglycerides². The referenced articles are hereby incorporated herein by reference. ¹Abe Y. El-Masri B. et al. Soluble cell adhesion molecules in hypertriglyceridemia and potential significance on monocyte adhesion. Arteriosler Thromb Vasc Biology 1998:18:723-731.²Ridker, Paul, Effects of n-3 Fatty Acid Therapy on Lipids and sCAMs—Inflammatory Markers, Pharmacotherapy and Clinical Trials, Lipids Online.org, posted: Oct. 3, 2001, reviewed Oct. 4, 2001.

EPA and DHA are also well known to those skilled in the art to reduce inflammation, decrease arrhythmias, decrease risk of sudden cardiac death and cardiac arrest.

Most of the clinical trials have used a more concentrated form of EPA−DHA as compared to the original non-concentrated fish oils which are commonly sold as nutritional supplements. In non-concentrated fish oil, approximately 20% to 30% of the fatty acids are EPA and DHA. In contrast, purified fish oil concentrate can contain from 60% to 95% EPA and DHA. Because of the process for concentrating EPA and DHA the chemical form of EPA−DHA may differ between non-concentrated fish oil and purified fish oil. The primary form of EPA and DHA in non-concentrated fish oil is triglycerides, whereas purified EPA and DHA may be mixtures of mono, di, and triglycerides, ethyl-esters, or salts of the fatty acids.

A significant number of clinical trials have been performed with a prescription omega-3 drug, formerly known as Omacor and now known as Lovasa™ (a trademark of Reliant Pharmaceuticals, Inc.). Lovasa is approved by the FDA for treating very high serum triglyceride levels (≧500 mg/dL) in adults. Lovasa contains 84% (weight) of combined EPA and DHA (46.5% EPA and 37.5% DHA) both in the ethyl-ester form. The medication is dispensed in 1 gram gelatin capsules and the recommended daily dose for treating very high triglycerides is 4 grams per day. Lovasa is based on U.S. Pat. Nos. 5,502,077 and 5,656,667, both of which are hereby incorporated herein by reference.

Lovasa has proven very effective in reducing triglycerides in individuals with very high triglyceride levels (by definition, >500 mg/dL) as shown in Table 1. Triglycerides were reduced by 51.6% (Treated group—Placebo group). It seems likely, however, that the reason the drug is limited by FDA for use on patients with very high triglycerides is because Lovasa concomitantly increased the low density (bad) cholesterol by 49.3%, a disadvantage that must be overcome by the advantages of the triglyceride reduction. Lovasa's manufacturer now recommends, in the package insert, using Lovasa with an HMG-CoA reductase inhibitor (statin) to overcome this disadvantage by reducing cholesterol with the statin.

TABLE 1
Clinical Trial with Lovasa (42 patients with very high triglyceride
levels (<500 mg/dl) dosage 4 g/day

| Parameter | % Change (Treated Group - Control Group |
|---|---|
| Triglycerides | −51.6 |
| Non-HDL-C | −10.2 |
| TC | −8.0 |
| VLDL-C | −40.8 |
| HDL-C | +9.1 |
| LDL-C | +49.3 |

These findings are consistent with the general consensus of the scientific community as published in Medline Plus, entitled “Omega-3 fatty acids, fish oil, alpha-linolenic acid” based on a professional level monograph edited and peer-reviewed by contributors to the Natural Standard Research Collaboration, Nov. 1, 2006. The source states “there is strong scientific evidence from human trials that omega-3 fatty acids from fish or fish oil supplements (EPA+DHA) significantly reduce blood triglyceride levels. Benefits appear to be dose-dependent. Fish oil supplements also appear to cause small improvements in high-density lipoprotein (“good cholesterol”); however, increases (worsening) in low-density lipoprotein levels (LDL/“bad cholesterol”) are also observed.” This finding is based on clinical trials using purified oils in the ethyl-ester form.

Another highly concentrated form of Omega-3 oils is disclosed in U.S. Pat. Nos. 6,689,812 and 7,119,118, both of which are hereby incorporated herein by reference. These patents disclose a pharmaceutical preparation comprising EPA in an appropriately assimilable form where of all the fatty acids present in the preparation at least 90%, and preferably at least 95%, are in the form of EPA and where less than 5%, and preferably less than 3%, are in the form of docosahexaenoic acid (DHA) The EPA is 96% in the ethyl-ester form. Such preparations are said to be for the treatment of any disorder except peripheral vascular disease and hyper-triglyceridaemia.

There is a need for an EPA/DHA composition which lowers triglycerides without concomitant increase in Low Density (bad) cholesterol while maintaining a high concentration of the combination of EPA and DHA (80%).

There is a need for a method of reducing triglycerides without concomitant increase in Low Density (bad) cholesterol with a combination of EPA and DHA at a high total concentration (over 70%).

There is a need for a method of reducing triglycerides without concomitant increase in Low Density (bad) cholesterol with a combination of EPA and DHA at a high total concentration (over 60%).

### SUMMARY

Provided herein are purified forms of Omega-3 fatty acids (over about 60% (by weight) EPA+DHA. In one aspect, a pharmaceutical composition is provided in which the active ingredients consist essentially of a mixture of fatty acids of which the mixture comprises at least about 60%, preferably about 75%, more preferably about 80% and most preferably about 90%, by weight of a combination of eicosapentaenoic acid (EPA) and docosahexaenoic acid (DHA). In some embodiments, the weight ratio of EPA:DHA is at least about 1:1 and preferably about 3:1 and more preferably about 4:1, and most preferably about 4.3:1. In some embodiments, at least about 60% of said combination of EPA and DHA is in the triglyceride form, with the balance, which is not triglycerides, comprising at least 90% mono and di-glycerides.

One exemplary composition comprises about 35% EPA, about 25% DHA and about 10% other omega-3 fatty acids, wherein the omega-3 fatty acids are at least 60% in the triglyceride form and the balance are about 90% mono and di-glycerides. One preferred composition is about 65% EPA and about 15% DHA and about 20% other omega-3 fatty acids, wherein the EPA and DHA are at least about 60% in the triglyceride form and the balance are at least about 90% mono and di-glycerides. Another preferred composition is about 75% EPA and about 15% DHA, wherein at least about 60% of the combination of DHA and EPA are in the triglyceride form and the balance is at least about 90% mono and di-glycerides.

The composition may conveniently be dispensed in liquid form or as a liquid filled gelatin capsules. The compositions are valuable for the treatment and prophylaxis of hypertriglyceridemia and hypercholesterolemia in human subjects in need of such treatment and/or prophylaxis. The composition is not limited for use by subjects having very high triglycerides and may be used as a prophylactic by subjects without significantly elevated triglyceride concentrations, without concern for increasing their LDL serum triglycerides. The preferred dosage is one to five grams per day, preferably two to four grams per day. Compositions when taken as directed preferably reduce serum triglyceride concentrations without a concomitant increase in LDL cholesterol concentration. In one embodiment, the composition increases serum High Density (good) cholesterol (HDL) concentrations. In one embodiment, the composition will decrease Very Low Density cholesterol (VLDL) concentrations. In one embodiments, the composition will reduce Lipoprotein(a) concentrations, apolipoprotein B concentrations and/or intermediate-density lipoprotein (IDL) cholesterol concentrations. Lipoprotein (a) and apoliproprotein B are components of LDL cholesterol, known as “heart attack cholesterol” and are important predictors of heart attack.

In some embodiments of the compositions a majority of the EPA and DHA are in their triglyceride form and the remainder comprises substantially mono and di-glyceride. In some embodiments, preferably at least about 90% and preferably about 95% of the EPA and DHA are either mono, di, or triglycerides. The results of a clinical trial showing the benefits of using the glyceridic forms of EPA and DHA are presented in Example 1. This work is based on an as yet unpublished study “Evaluation of Lipid Profiles, Inflammatory markers and the use of Omega-3 EFA in Professional Football Players”, PI-Anthony Yates, University of Pittsburgh Medical Center”.

The composition used in the University of Pittsburgh Study was less concentrated than Lovasa (60% EPA and DHA vs. 84% for Lovasa) and the daily dosage was lower (2.2 grams per day vs.4 grams per day of EPA+DHA). The omega-3 fatty acids were about 60% in the triglyceride form with the balance being mono and di-glycerides as compared to essentially 100% in the ethyl-ester form in the Lovasa trial. The source of the composition used in the University of Pittsburgh study was ProOmega®, a Registered Trademark of Nordic Naturals, Inc. The serum triglyceride reduction in the treated subjects was about the same in both trials, however the LDL (bad) cholesterol did not increase at all in the University of Pittsburgh trial and the increase in HDL (good) cholesterol was greater in these trials using the triglyceride form of omega-3s than in the ethyl ester (Lovasa) trials, as was the (Very Low Density) VLD cholesterol. It therefore appears that the triglyceride forms of EPA and DHA are more beneficial for serum triglyceride and cholesterol treatment than the ethyl ester form of EPA and DHA, and significantly will not need to be combined with statins for use with persons having less than very high triglycerides. Omega-3 Fatty acids are found in nature in the triglyceride form (a glycerol with three fatty acids attached). This is the only form that could have been ingested by man and his ancestors during evolutionary times. The natural triglyceride form as found in raw fish oil can not be readily separated as it occurs into purified EPA/DHA mixtures by ordinary means such as distillation or crystallization, because the fatty acids are non-uniformly distributed among the triglyceride molecules. There are very few, if any, single triglyceride molecules which are composed of either three EPAs or three DHAs. Typically, there is a DHA, an EPA, and another fatty acid in a triglyceride molecule. So in order to purify fatty acids to increase the proportion of EPA, DHA, or the total fraction of omega-3's, it is necessary to hydrolyze the triglycerides to remove at least some fatty acids from the glycerol.

The triglycerides may be converted by any method known to one skilled in the art without limitation. For example, the triglycerides may be converted by lipase-catalyzed esterification or lipase catalyzed acidolysis with ethyl or lauryl alcohol, which can selectively leave the highest amount of EPA and DHA bonded to glycerols and remove other components, leaving EPA and/or DHA as mono or di-glycerides. The mono and di-glycerides can then be separated into fractions with different EPA/DHA ratios, by methods familiar to those skilled in the art such as multiple stage vacuum distillation and/or fractional crystallization in urea. Advantageously, the purified EPA and DHA esters, after concentration, can be reattached to glycerol molecules using enzymatic reacylation to recreate glycerides which are otherwise identical to the original natural triglycerides, except that they are more concentrated in EPA and DHA combined, and they may also have a different ratio of EPA:DHA than the original fish oil. In some embodiments, at least 60% of the omega-3 fatty acids, and preferably 70% or more, are converted to the triglyceride form in the reacylation process. The process may be successively repeated with addition of additional catalyst and/or enzyme and additional EPA and DHA until the desired specification proportions are met. About 60% of triglycerides can be made in the first pass of reacylation, with most of the remainder of the product being mono and di-glycerides.

It is an object of the invention to provide an omega-3 fatty acid formulation which when ingested by humans lowers triglycerides and either lowers LDL (bad) cholesterol, or at least does not raise it.

It is a further object of the invention to provide an omega-3 fatty acid formulation which when ingested by humans lowers triglycerides and lowers LDL cholesterol, while increasing HDL (good) cholesterol.

### DETAILED DESCRIPTION OF THE INVENTION

In one aspect provided herein a pharmaceutical composition in which the active ingredients consist essentially of a mixture of fatty acids, which mixture comprises at least about 60%, preferably about 75% more preferably about 80%, and most preferably about 90% or more, by weight of a combination of eicosapentaenoic acid (EPA) and docosahexaenoic acid (DHA). In some embodiments, the weight ratio of EPA:DHA is at least about 1:1, preferably about 3:1 and more preferably about 4.3:1. In some embodiments, at least about 60% and more preferably about 70% or more of said combination of EPA and DHA is in the triglyceride form as opposed to the ethyl-ester or other form. One exemplary composition has about 35% EPA, about 25% DHA and about 10% other omega-3s, wherein the omega-3s are at least about 60% in the triglyceride form and the balance is at least about 90% mono and di-glycerides. A preferred composition comprises about 65% EPA and about 15% DHA and about 20% other omega-3 fatty acids, wherein the combination of EPA and DHA are at least about 60% in the triglyceride form, preferably at least about 75% or more, with the balance being at least about 80% and more preferably about 85% or more in the mono and di-glyceride forms. Another preferred composition is about 75% EPA and about 15% DHA, wherein at least about 60% of the combination of DHA and EPA is in the triglyceride form and the balance is at least about 90% mono and di-glycerides. The composition may be dispensed in liquid form or as a liquid filled gelatin capsule.

In another aspect, provided herein is an improved method for the treatment or prophylaxis for hypertriglyceridemia or hypercholesterolemia in human patients without concomitant increase in LDL cholesterol. The preferred dosage is about one to about five grams per day, preferably about two to about four grams per day taken orally. The treatment reduces serum triglycerides, increases HDL (good) cholesterol, and decreases or at least does not increase LDL (bad) cholesterol.

The composition is preferably administered orally and may be delivered as a liquid or as liquid filled capsules, such as gelatin capsules, vegetable starch-based capsules, alginate capsules, or equivalents thereof.

Example 1 shows the results of a clinical trial with a pharmaceutical composition as described herein This composition comprised about 35.5% EPA and 25.5% DHA and about 10% other Omega-3s with about 60% in the triglyceride form with most of the balance mono and diglycerides.

### EXAMPLE 1

### University of Pittsburgh Trials (20 Treatment Patients, 16 Control Patients, 8 Months Duration) Oil Composition: 35% EPA, 25% DHA and 10% Other Omega-3s- 60% in Triglyceride Form

| | % Change (Treated Group - Control Group |
|---|---|
| Triglycerides | −60.6 |
| Non-HDL-C | −10.7 |
| TC | −0.96 |
| VLDL-C | −45.4 |
| HDL-C | +16.1 |
| LDL-C | −1.6 |

Serum triglycerides were reduced by 60.6% and the LDL Cholesterol did not increase at all compared to a 49.3 increase in the Lovasa trial (see Table 1, above). Further, VLDL cholesterol significantly (bad) decreased and HDL (good) cholesterol significantly increased. The VLDL and LDL cholesterol are high risk factors for developing atherosclerosis, a precursor to blood clots, heart attacks, and stroke. It is also noteworthy that two other cholesterol forms which are components of LDL cholesterol, were also reduced. The IDL cholesterol was reduced 46.9% and the Lipoprotein (a) was reduced by 8.5%. These are generally thought to be largely inherited factors which are highly predictive of heart attack risk (they are known as “Heart Attack Cholesterol”).

In some advantageous embodiments, about 60% or more of the EPA−DHA combination are in the triglyceride form. In non-concentrated fish oil, approximately 20% to 30% of the fatty acids are EPA and DHA. In contrast, highly purified fish oil concentrates can contain from 60% to 95% EPA and DHA. In non-concentrated fish oil (the oil extracted directly from the fish) the EPA and DHA are in the triglyceride form which is the form found in nature. When the EPA and DHA are concentrated it is necessary to remove some or all of the fatty acids from the triglyceride in order to concentrate the EPA and DHA. One way of doing this is hydrolysis in ethanol (ethyl esterification), which will remove most, or all of the fatty acids as ethyl-esters. The esters can then be concentrated into EPA rich and DHA rich streams by vacuum distillation. These streams can be combined together with glycerol and a lipase enzyme such as Candida antarctica lipase, Chromobacterium viscosum lipase, or others where the esters are reattached to the glycerol to make a mixture of approximately 60% triglycerides with the balance mono and diglycererides. To make higher levels of triglycerides the hydrolysis may be conducted with selective hydrolysis or alcoholysis using ethanol or lauryl alcohol and lipase enzymes as were used above for reattachment of the esters to glycerols. This leaves a mixture of ethyl esters and mono and diglycerides now enriched in EPA and DHA. The ethyl esters can be distilled into an EPA rich stream, and the mixture reacylated by adding additional enzymes and reacting. One can get to about 75%-90% or more triglycerides by adding more enzymes and repeating the reacylation step as is required to meet the specification. The reaction times for reacylation are long, possibly 24 hours at 120° C. per cycle.

The compositions are effective for reduction of triglycerides without an increase in LDL cholesterol. It is therefore safe to use for people with less than high cholesterol and even as a prophylactic for people with normal or slightly elevated cholesterol. It will also reduce VLD cholesterol substantially. This VLD cholesterol is high risk factors for developing atherosclerosis, a precursor to blood clots, heart attacks, and stroke. The recommended dosage is two to four grams per day orally, either as a liquid or a liquid filled capsule.

How to Make the Compositions of the Invention

1. Removal of Free Fatty Acids

Raw fish oil in the natural triglyceride molecular preferably from anchovies and sardines which contain about 18% EPA and 12% DHA is heated to 60° C. to decrease viscosity. Sodium oxide is added to bind with free fatty acids in the oil. The mixture is moved to a separator where Sodium oxide bound to free fatty acids (soap) floats to the top and is removed.

The oil is then moved to a second separator where warm water is preferably added to help remove traces of Sodium oxide, as Sodium oxide partitions to water, yet does not interact with the fish oil.

Citric acid may then be added to support splitting the oil from the combination of water and Sodium oxide. The oil is then cooled to 30° C. to protect it from oxidation.

2. Stripping and Purification

Oil is moved to a separate stripping tank, and heated to 200° C. Ethyl esters can be added to support the removal of impurities, which bind to ethyl esters. Impurities such as dioxins, heavy metals, pcbs, fire retardants, furans and others evaporate and are drawn to the middle of the tank where a refrigerating element cools them down and drain them. The added esters are also removed to with the impurities.

3. Esterification

The oil is moved to an esterification tank. Ethanol and sodium metal are added. Sodium metal is a catalyst for breaking off fatty acid strands from the glycerol backbone of the TG fatty acid molecule, the free fatty acids then combined with ethanol to form ethyl esters. Water can be added to bind to sodium metal, where the combination of water and sodium metal can be removed.

4. Molecular Distillation

The oil is then moved to a distiller where it is heated to about 120° C. under vacuum. Mono esters and shorter carbon chain molecules move to the middle where they are cooled and drained, leaving longer carbon chains remaining as a concentrate. The process typically increases the key fatty acids by 100% during the first distillation; typically between 30-50% during the second distillation. The process can be repeated, although preferably the process is ideally only repeated once, as when oils undergo heat which can produce oxidation and degradation of the fatty acids in general. Oil waste is also increasing with repeated distillation, making the process less economical.

5. Reesterification (Reacylation)

The oil is then moved to a reesterification tank where the ethyl ester molecules are reconverted to the triglyceride form, which is the natural form of that fatty acid molecule. 98% of fats ingested by humans are in this natural triglyceride form.

The esterification process takes place under low vacuum at about 80° C. Glycerol is added to form the backbone of the glyceride molecules. Nitrogen can be added from the bottom of the tank to cause oil movement. Lipase enzymes are added as catalysts to facilitate the fatty acids binding to glycerol. The vacuum in the distillation tank removes the ethanol which was previously bound to the fatty acids. The enzymes used are lipases produced from bacteria or yeast. Perhaps the most effective enzymes are Candida antarctica lipase, and Chromobacterium Viscosum Lipase; other enzymes that can be used effectively are Psuedomonas. Mucor miehei, and Candida Cylindracea as well as other enzymes may also be used.

The reesterification process typically takes 24 hours, at which point the triglycerides typically reaches 60-65%, the remaining glycerides being diglycerides and monoglycerides. Around 3% of the fish oil will remain as ethyl esters, which can be removed together with the ethanol. Adding additional enzymes and/or continuing the enzymatic process can produce triglyceride molecule concentration of up to 99%. The 60-65% level is probably optimum from an economic point of view. There is little additional benefit in pharmaceutical performance for triglyceride/cholesterol control in going to higher concentrations.

6. Winterization

The oil in triglyceride form is then moved to a cooling tank at 0° C., where saturated fats, in particular stearic acid are crystallized. The pulp is then pumped to a filter press, where the crystals are removed, essentially removing the vast majority of saturated fats from the oil. Depending on the amount of saturated fats in the oil, approximately 5-10% of the oil is lost during this process.

7. Bleaching

The oil is then removed to a bleaching tank at 60° C., where bleaching earth or bentonite earth is added to the oil. Any water in the oil evaporates due to the temperature. Any remaining impurities (trace minerals etc) in the oil attach to the bentonite earth. The oil is then run though a bentonite earth filter to remove the bentonite earth together with the impurities.

8. Deodorization

Although not a necessary step, it is advantageous to move the oil to a deodorization tank. The tank contains low vacuum at 120° C. Steam is added at the bottom of the tank, which connects to color and odor molecules (oxidated matter, peroxides), which again travel into the vacuum system and into a residue container. This process gives the oil a neutral color with virtually zero taste and odor.

9. Mixing.

The oil is then moved to a separate storage tank. Depending on the concentration of EPA and DHA desired, various batches can be mixed to yield the concentration desired for the final product.

10. Antioxidants, in particular rosemary and mixed tocopherols can be added to the final oil to dramatically reduce the oxidation process.

11. Drumming

The oil is then drummed in stainless steel drums for storage and topped off with nitrogen to remove oxygen and minimize the potential for oxidation.

What is claimed is:

1. A method for the treatment or prophylaxis of hypertriglyceridemia and hypercholesterolemia without concomitantly increasing LDL-serum cholesterol, in a human subject requiring such treatment, which method comprises orally administering to the subject an effective amount of a pharmaceutical composition in which the active ingredients comprise a mixture of fatty acids, wherein said mixture comprises at least about 60% by weight of a combination of eicosapentaenoic acid (EPA) and docosahexaenoic acid (DHA) in a weight ratio of EPA:DHA of from about 1.4:1 to about 5:1, wherein said combination is at least about 60% in the triglyceride form of the fatty acids and the balance is at least about 80% of mono and di-glycerides.

2. The method of claim 1, wherein the treatment results in reduction of serum IDL cholesterol concentration

3. The method of claim 1, wherein the treatment results in reduction of serum Lipoprotein(a) concentration.

4. The method of claim 1, wherein the treatment results in reduction of serum VLDL concentration.

5. The method of claim 1, wherein the treatment results in an increase of serum HDL concentrations.

6. The method of claim 1, wherein the treatment results in reduction of serum apolipoprotein (B) concentrations.

7. The method of claim 1, wherein said ratio of EPA:DHA is in the range between 2:1 and 5:1.

8. The method of claim 1, wherein the active ingredients comprise a mixture of fatty acids, wherein said mixture comprises at least about 75% by weight a combination of eicosapentaenoic acid (EPA) and docosahexaenoic acid (DHA).

9. The method of claim 1, wherein the active ingredients comprise a mixture of fatty acids, wherein said mixture comprises at least about 80% by weight eicosapentaenoic acid (EPA) and docosahexaenoic acid (DHA).

10. The method of claim 1, wherein the active ingredients comprise a mixture of fatty acids, wherein said mixture comprises at least about 90% by weight eicosapentaenoic acid (EPA) and docosahexaenoic acid (DHA).

11. The method of claim 1, wherein the combination comprises about 35% (weight) EPA and about 25% ( weight) DHA.

12. The method of claim 1, wherein the combination comprises about 55% EPA and about 20% DHA.

13. The method of claim 1 wherein the combination comprises about 65% EPA and about 15% DHA

14. The method of claim 1, wherein the combination comprises about 65% EPA and about 25% DHA

15. A pharmaceutical composition, comprising of a mixture of fatty acids, wherein said mixture comprises at least about 75% by weight a combination of eicosapentaenoic acid (EPA) and docosahexaenoic acid (DHA) in a weight ratio of EPA:DHA of from 1.4:1 to about 5:1, wherein said combination is at least about 60% in the triglyceride form of the fatty acids and the balance is at least 80% mono and di-glycerides.

16. The pharmaceutical composition of claim 15, wherein the weight ratio of EPA:DHA is in the range from about 2:1 to about 5:1.

17. The pharmaceutical composition of claim 15, wherein said mixture comprises at least about 80% by weight of a combination of eicosapentaenoic acid (EPA) and docosahexaenoic acid (DHA).

18. The pharmaceutical composition of claim 15, wherein said mixture comprises at least about 85% by weight of a combination of eicosapentaenoic acid (EPA) and docosahexaenoic acid (DHA).

19. The pharmaceutical composition of claim 15, wherein said mixture comprises at least about 90% by weight a combination of eicosapentaenoic acid (EPA) and docosahexaenoic acid (DHA).

20. The pharmaceutical composition of claim 15, wherein the combination comprises about 60% EPA and about 15% DHA.

21. The pharmaceutical composition of claim 15, wherein the combination is about 60% EPA and about 20% DHA.

22. The pharmaceutical composition of claim 15, wherein the combination is about 65% EPA and about 15% DHA.

23. The pharmaceutical composition of claim 15, wherein the combination is about 65% EPA and about 25% DHA.
